(19) United States
(12) Patent Application Publication
(10) Pub. No.: US 2006/0069394 A1
(43) Pub. Date: Mar. 30, 2006

(54) Device for guiding a drilling tool

(76) Inventors: Andreas Weiler, Berlin (DE); Sascha Berberich, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 11/239,267

(22) Filed: Sep. 29, 2005

(30) Foreign Application Priority Data
Sep. 29, 2004 (DE) 10 2004 048 042.7

(51) Int. Cl. A61B 17/60

(52) U.S. Cl. 606/96

(57) ABSTRACT

A device (10) serves for guiding a drilling tool (56) for making a bore (58) into a bone (50), intersecting an already existing first bore (54). The device (10) has a first rod-shaped body (12), the first end of which can be introduced into the already existing first bore (54) in the bone (50). Its second end is provided with an arm, which bears a guide (30) for the drilling tool (56), the longitudinal axis of which intersects the rod-shaped body (12) in the region of its first end. The arm has an arcuate portion (26), along which the guide (30) for the drilling tool (56) can be moved. An opening (16) is provided in the rod-shaped body allowing the drilling tool (56) to pass said rod-shaped body.

This application claims priority of German Patent Application No. 10 2004 048 042.7 filed on Sep. 29, 2004.

### BACKGROUND OF THE INVENTION

The invention relates to a device for guiding a drilling tool for making a bore into a bone, said bore intersects an already existing first bore, said device having a first rod-shaped body, a first end of which can be introduced into the already existing first bore in the bone, a second end of which is provided with an arm which bears a guide for the drilling tool, a longitudinal axis of the drilling tool intersects the rod-shaped body in the region of its first end, said arm having an arcuate portion, along which the guide for the drilling tool can be moved, the centre point of an envelope of the arcuate portion lying at the point of intersection of the longitudinal axis of the drilling tool with the rod-shaped body.

### PRIOR ART

A device of this type is known from U.S. Pat. No. 5,688,284.

A device of this type is used for the fixing of a ligament implant inserted into a bone, usually in the region of the knee joint.

For this purpose, a drilling tool is used to introduce a first bore, which is formed as a blind-hole bore, into the bone, for example into the femur. The ligament implant is pushed into this blind-hole bore.

For fixing this ligament implant, a cross bolt is introduced, driven transversely into the ligament or into a loop in the ligament, so that the ligament is fixed by this cross bolt, also known as a cross pin, against being pulled out of the first bore.

To introduce the cross pin, it is necessary to introduce a further, second bore, which is aligned in such a way that it intersects the first bore, that is to say runs transversely, usually at right angles, in relation to the latter.

Since the position of the blind-hole bore in the bone is not visible to the operating surgeon from this transverse direction, auxiliary devices or sighting devices were created in order to make the second transverse bore for introduction of the cross pin in exactly the right position or as targeted by the sighting, i.e. in such a way that the second bore meets the first bore.

In the case of U.S. Pat. No. 5,688,284, mentioned at the beginning, a rod-shaped body, which can be inserted into the already existing first bore, is provided for this purpose. At its second end, lying outside the bone, the rod-shaped body is provided with a laterally projecting arm.

The end section of said arm is designed as an arcuate portion, along which the guide for the drilling tool can be moved.

The centre point of an envelope of the arcuate portion lies at the point of intersection of the longitudinal axis of the drilling tool with the rod-shaped body.

When performing the bore with the drilling tool, a tip of the drilling tool meet a target depression provided in the rod-shaped body with said design. A bore continuing beyond the rod-shaped body cannot perform.

It is therefore an object of the invention to allow a bore beyond the rod-shaped body.

### SUMMARY OF THE INVENTION

According to the invention, an opening is cut out in the rod-shaped body in the region of the point of intersection, said drilling tool passes said rod-shaped body through said opening.

This measure has the considerable advantage that the transverse bore can be performed in a way, passing through the rod-shaped body, and continuing beyond the rod-shaped body.

For a cross pin it is necessary that it extends on both sides in the transverse direction in relation to the first bore, in order that it is anchored in the bone on both sides. For this purpose it is helpful to continue the transverse bore correspondingly in the drilling direction also on the opposite side in relation to the first bore.

The way in which it is now proposed for an opening to be structurally provided in the rod-shaped body allows the making of a transverse bore which extends beyond the first bore by a single operation.

In one configuration, the opening in the rod-shaped body is formed as a continuous bore, through which the drilling tool can be guided in the various angular positions.

This measure has the advantage that the drilling tool can pass through the opening in the rod-shaped body in all possible angular positions of the guide of the drilling tool along the arcuate portion.

In a further configuration of the invention, the opening is provided with bevels, which serve as a running-in guide for the tip of the drilling tool.

This measure has the significant advantage that, as a result, the drilling tool can be guided in such a way that it is directed at the target point.

Should there be slight deviations or deflections on account of anatomical conditions during the driving-in of the second drilling tool, the bevels guide the tip of the drilling tool in a targeted manner into the opening, through the latter, so that the bore can also be continued in a targeted manner on the opposite side.

In a further configuration of the invention, the arcuate portion is movably attached to the arm.

This measure further increases the flexibility to the extent that not only can the guide be moved along the arcuate portion, but that this arcuate portion itself can also be additionally moved.

In one configuration, it is provided that the arcuate portion is pivotably attached.

This pivoting capability allows virtually an entire area of a spherical portion to be selected as a sighting area which can be covered by the extent of the portion.

In a further configuration, the arcuate portion is displaceably attached.

This measure further increases the flexibility to the extent that the arcuate portion can also be additionally displaced along its circular line.

If this measure is applied in conjunction with the previously mentioned measure of the pivoting capability, a very large spherical surface portion can be covered by a relatively short circular portion, so that virtually the entire available region can be targeted, in particular in the region of the femur or the tibia.

In the event that a second cross pin is to be fitted or the point of intersection of the axis of the drilling tool with the first bore is to be changed in the longitudinal position, the arcuate portion as such can be displaced along the arm.

In a further configuration of the invention, the portion has an angular scale.

As a result, it is possible to establish or document the exact angular position of the channel for pushing in the cross pin in relation to the longitudinal axis of the first bore.

In a further configuration of the invention, the arm is formed as a U-shaped bow, the one leg of which forms the rod-shaped body.

This measure has the advantage that a very stable, slender configuration of the basic body which is also simple to produce is possible. One leg of which can be pushed with its first end into the already existing first bore for the introduction of the ligament implant. Subsequently, the operating surgeon can carry out the desired manipulation for placing, aligning and sighting the drilling tool.

In a further configuration of the invention, the arcuate portion is attached to the end of the U-shaped body lying opposite the first rod-shaped body.

This measure has the advantage that the U-shaped body can be handled well by the operating surgeon and that the outer, second end of the U lies at the height of the first, inner end of the U, pushed into the first bore. In this region of the “U”, there are of course the widenings of the femur and tibia that are present in the joint, which are embraced by the arcuate portion which bears the guide.

It goes without saying that the features stated above and those still to be explained below can be used not only in the specified combinations but also in other combinations or on their own without departing from the scope of the present invention.

### BRIEF DESCRIPTION OF THE DRAWINGS

The invention is described and explained in more detail below on the basis of several selected exemplary embodiments in conjunction with the accompanying drawings, in which:

FIG. 1 shows a side view of a first exemplary embodiment of the device according to the invention in a highly schematized form,

FIG. 2 shows a side view comparable to FIG. 1 in another setting or configuration of the arcuate portion,

FIG. 3 shows the device according to the invention from FIG. 1 after making the transverse bore for the cross pin by the drilling tool in a perspective and highly schematized form,

FIG. 4 shows a greatly enlarged view of the first end of the rod-shaped body in the form of a detail in the region of the opening for guiding through the drilling tool, various angular positions of the drilling tool being represented,

FIG. 5 shows a representation comparable to the representation of FIG. 3, with a further exemplary embodiment of a device according to the invention, and

FIG. 6 shows an application of the device represented in FIG. 5 in another angular position of the drilling tool and in the case of another surgical technique.

### DETAILED DESCRIPTION OF PREFERRED EMBODIMENTS

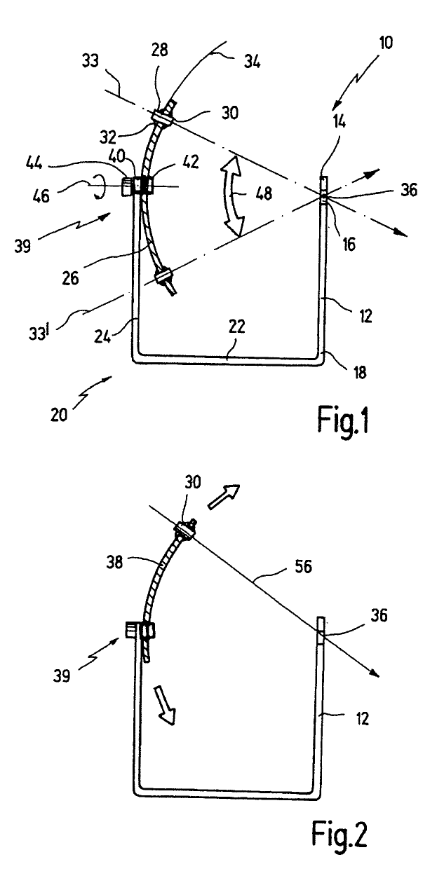

A first embodiment, represented in FIGS. 1 to 4, of a device according to the invention is provided in its entirety with the reference numeral 10.

The device 10 has a longitudinal extended rod-shaped body 12 with a circular cross section.

In the region of its first end 14 there is an opening 16 in the form of a continuous bore, as described later in more detail in conjunction with FIG. 4.

The second end 18, lying opposite from the first end 14, continues in an angled-away arm 20 projecting approximately at right angles.

The arm 20 comprises a cross bar 22 and an upright leg 24. As can be seen from FIG. 1, the arm 20 is formed in such a way that, together with the rod-shaped body 12, an approximately U-shaped body with identical legs is created, which can be produced from one and the same rod-shaped material by appropriate bending operations.

Attached to the outer end region of the upright leg 24 is an arcuate portion 26. The arcuate portion 26 bears a guide 28 for a drilling tool.

The guide 28 is formed as a guiding sleeve 30, which can be displaced back and forth along the arcuate portion 26. A fixing screw 32 serves the purpose of making this displacing operation possible and fixing the guiding sleeve 30 in a specific position. The guiding sleeve 30 is aligned in such a way that its centre longitudinal axis 33 is aligned in the direction of a point 36, which lies approximately in the centre of the opening 16 in the rod-shaped body 12. This point represents the centre point 36 of an envelope 34 of the arcuate portion 26. If, as described in more detail later, a drilling tool is pushed into the guiding sleeve 30, said tool is aligned along the longitudinal axis 33 of said sleeve, targeted at the opening 16 in the first end 14 of the rod-shaped body 12, as indicated by the double-dash-segmented line.

Loosening of the fixing screw 32 allows the guiding sleeve 30 to be moved downward for example in the representation of FIG. 1, until the alignment of the drilling tool corresponds to the alignment indicated by the dash-segmented longitudinal axis 33′.

In all these different angular positions along the arcuate portion 26, the centre longitudinal axis of the drilling tool is aligned exactly with the opening 16.

This therefore creates here in this plane the pivoting range for the drilling tool indicated by the double-headed arrow 48.

The arcuate portion 26 is pivotably attached to the leg 24 by an arresting means 39.

The arresting means 39 has two clamping jaws 40 and 42, which can be released or fixed by means of a clamping screw 44. With the arresting means 39 released, the arcuate portion 46 can be pivoted about an axis 46 out of the plane of the drawing of FIG. 1 and arrested in a corresponding desired angular position. The degree of freedom of the alignments of the longitudinal axes 33, 33′ of the drilling tool which is pushed into the guiding sleeve 30 consequently lies within a conical body.

If it is wished to target a different point on the rod-shaped body 12, the arcuate portion 26 can be displaced along the leg 24. There can then be further openings in the rod-shaped body 12, or there is a longitudinally running slotted opening unit in it, so that the drilling tool can pass through the body 12.

In FIG. 2 it is shown that, by releasing the arresting means 39, the arcuate portion can not only be turned about the axis 46 but also be displaced or moved in the direction of the envelope 34. By corresponding displacement, as represented for example in FIG. 2, it is possible to increase the degree of freedom still further in its extent, so that a significantly greater conical angular range can be covered.

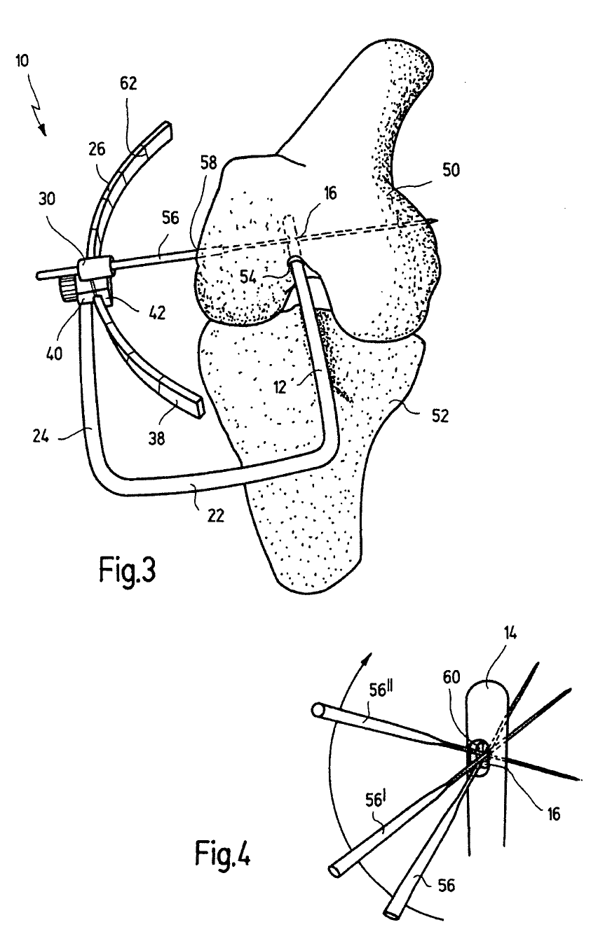

The purpose and advantage of this flexible movement of the guiding sleeve 30 and of the arcuate portion 26 in practical use is now to be explained on the basis of FIGS. 3 and 4.

In FIG. 3, a knee joint, to be precise a femur 50 and tibia 52, is represented in a highly schematized form. On the femur 50 it is intended to anchor a ligament, which is to serve as a replacement for a defective cruciate ligament.

For this purpose, a first bore 54 was introduced into the femur 50 in the form of a blind-hole bore. This can be continued by a bore of small diameter up to the outer side, in order for example to lead through threads for securing or drawing in the ligament. To introduce this first bore 54 as targeted by the sighting, it is possible for example to use a sighting device, as known from U.S. Pat. No. 5,350,383.

In FIG. 3 it is shown how the end region of the rod-shaped body 12 of the device 10 is pushed into this first bore 54. For this purpose, the outside diameter of the rod-shaped body 12 is chosen such that it can be pushed into the first bore 54.

As can be seen from FIG. 3, the opening 16 in the rod-shaped body 12 lies within the first bore 54.

The arcuate portion 26 lies in a plane which is defined by the U-shaped body, made up of the rod-shaped body 12 and the arm 20. The guiding sleeve 30 lies at the height of the arresting means 39 and a drilling tool 56 was pushed in through the opening of the guiding sleeve 30 and driven into the femur 50 so far as to create a second bore 58, which runs transversely in relation to the first bore 54, intersects the latter and extends beyond the first bore 54. In the embodiment represented, the drilling tool 56 is driven through the entire femur 50. By the aforementioned alignment, the drilling tool 56 was led exactly through the opening 16 in the rod-shaped body 12, in order to provide the second bore 58.

After pulling off the drilling tool 56, a so-called cross pin is driven into the second bore 58, serving the purpose of anchoring or fixing a ligament previously introduced into the first bore 54.

It can be seen from the enlarged representation of a detail in FIG. 4 that the opening 16 has bevels 60.

As a result, the drilling tool 56 is smoothly directed or guided into the opening 16 in every angular position.

Depending on the extent of the length of the circular portion 26, the opening is formed as a slotted opening with a correspondingly peripheral bevel 60. It is possible to correct small erroneous deviations when driving in the drilling tool 56 by the latter being introduced into the opening 16 in a targeted manner by the bevels 60.

It can be seen from FIG. 3 that an angular scale 62 is provided on the circular portion 26, to allow the exact setting or documentation of the displacement of the guide 30. For example, the position represented in FIG. 3 can be defined as the 0° position and upward deviations defined as positive degrees of deviation and downward deviations defined as negative degrees of deviation.

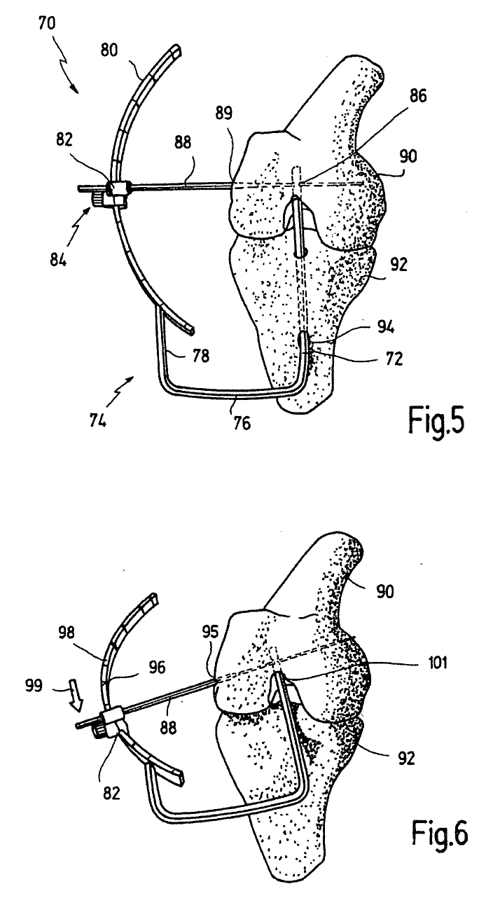

In the case of a further embodiment, represented in FIGS. 5 and 6, of a device 70 according to the invention, a first rod-shaped body 72, which is connected to an arm 74, is likewise provided. Here, too, the arm 74 has a cross bar 76, which goes over into an upright leg 78, which runs approximately parallel to the rod-shaped body 72. Firmly attached, for example welded, to the outer end of the leg 78 is the arcuate portion 80. The leg 78 is significantly shorter than the rod-shaped body 72.

On the arcuate portion 80, a guide 82 for a drilling tool 88 is again displaceably mounted. An arresting means 84 allows the guide 82 to be displaced and fixed along the arcuate portion 80. Here, too, the alignment of the guide 82 is again such that a drilling tool 88 pushed through it is aligned exactly with an opening 86 in the rod-shaped body 72, as previously described.

In FIG. 5 it is shown that the rod-shaped body 72 has been pushed into a first bore 94, which extends both through the tibia 92 and into the femur 90.

Represented in FIG. 6 is the use of the device 70 in the case of a somewhat different surgical technique, which is similar to that described in FIG. 3; here, therefore, the first bore 101 is merely driven into the femur 90. The guide 82 has in this case been displaced downward along the circular portion 80 in comparison with the representation of FIG. 5, for example by −20°, as is indicated by the arrow 99. Here, too, a corresponding scale 96 allows this to be performed in the right position. As can be seen, the arcuate portion 80 is formed as a rail 98 with a rectangular cross section. By releasing the arresting means or its adjusting screw, the clamping jaws between which the rail 98 is mounted are opened somewhat, so that the guide 82 can be correspondingly displaced. Once the desired position is reached, the arresting means is fixed and the drilling tool 88 can be pushed in.

This shows that a device according to the invention can respond very flexibly to the anatomical conditions, for example if, in the region of the femur, there happen to be corresponding defects or gaps which do not permit appropriate placement of the drilling tool.

As described above, after introduction of the second bore 95, a corresponding cross pin is then driven in, in order to fix the ligament or the ligament replacement which has in the meantime been introduced into the first bore 94.

What is claimed is:

1. A device for guiding a drilling tool for making a bore into a bone, said bore intersecting an already existing first bore, comprising
a first rod-shaped body, a first end of which can be introduced in an already existing first bore in a bone, a second end of said rod-shaped body is provided with an arm bearing a guide for a drilling tool, a longitudinal axis of said drilling tool intersects said rod-shaped body in a region of its first end, and
said arm having an arcuate portion, along which said guide for said drilling tool can be moved, a centre point of an envelope of said arcuate portion lying at a point of intersection of said longitudinal axis of said drilling tool with said rod-shaped body,
wherein an opening is cut out in said rod-shaped body in a region of said point of intersection, said drilling tool passes said rod-shaped body through said opening.

2. The device of claim 1, wherein said opening is formed as a continuous bore, through which said drilling tool can be guided in various angular positions.

3. The device of claim 1, wherein said opening is provided with bevels serving as a running-in guide for a tip of said drilling tool.

4. The device of claim 1, wherein said arcuate portion is moveably attached to said arm.

5. The device of claim 1, wherein said arcuate portion is pivotably attached to said arm.

6. The device of claim 1, wherein said arcuate portion is displaceably attached to said arm.

7. The device of claim 1, wherein said arcuate portion has an angular scale.

8. The device of claim 1, wherein said arm is formed as a U-shaped bow, one leg of which forms said rod-shaped body.

9. The device of claim 8, wherein said arcuate portion is attached to said U-shaped body lying opposite to a first end of said rod-shaped body.
